(19) United States
(12) Patent Application Publication
(10) Pub. No.: US 2016/0367201 A1
(43) Pub. Date: Dec. 22, 2016

(54) SYSTEM AND METHOD FOR PASSIVE EVENT DETECTION SYSTEM FOR TRAUMATIC EVENT

(71) Applicants: Joshua Daniel Korsower, Sterling, VA (US); Albert Fenwick Fleury, III, Chevy Chase, MD (US)

(72) Inventors: Joshua Daniel Korsower, Sterling, VA (US); Albert Fenwick Fleury, III, Chevy Chase, MD (US)

(21) Appl. No.: 15/189,799

(22) Filed: Jun. 22, 2016

Related U.S. Application Data
(60) Provisional application No. 62/182,782, filed on Jun. 22, 2015.

(51) Int. Cl. A61B 5/00; A61B 5/11; A61B 5/0205

(52) U.S. Cl. CPC A61B 5/7282; A61B 5/0205; A61B 5/1123; A61B 5/746; A61B 5/02438

(57) ABSTRACT

Systems and methods of passive and active alert system for individuals to detect traumatic events using physiological and environmental factors. Exemplary methods include receiving sensor data associated with the individual from a plurality of sensors of a monitoring device and determining whether the sensor data satisfies one or more trigger conditions. For each of the trigger conditions satisfied, one or more messages are sent to at least one of the patient monitoring device and/or an external computing device for analysis. Satisfaction of one or more of the trigger conditions may indicate the individual has been incapacitated and is in need of assistance. The sensor data may have been collected from a heart rate sensor and/or an accelerometer. In some embodiments, the trigger conditions are defined by the individual.

### FIELD

The present invention, a passive event detection system, is directed generally to systems and methods for monitoring individuals need for assistance, and more particularly, to methods and systems for detecting individuals who are incapacitated and in need of assistance.

### BACKGROUND

Elderly individuals face an increasing likelihood of a traumatic event which could render the individual incapable of calling for help, for example a fall which renders them unconscious or immobile. Clearly, those individuals living alone are at an even higher risk of being unable to communicate with someone in the event of such an event.

Physically impaired and/or mentally impaired individuals may be at an increased risk of injury, and may be at an increased risk of being unable to communicate or operate an active alert upon such an injury. This group includes individuals who may be permanently impaired, or temporarily impaired, for example those recovering from an injury or surgery.

Individuals who enjoy adventurous or “risky” activities on their own, such as biking, running or rock climbing, face a larger risk of being injured.

For many injuries, it is imperative to get appropriate care and medical attention as soon as possible. Early intervention often is the difference between life and death. With the baby boomer generation entering into the older years, the need for a passive alert system for non-geriatric populations is extremely high.

What is needed is a new way to identify the occurrence of an event that may have caused traumatic injury to the user, while minimizing false-positive event detections. What is needed is technology that fosters peace of mind among at-risk individuals and their loved ones. What is needed is a passive event detection system that would output an event notification when a user suffers a traumatic injury.

### SUMMARY

Embodiments described herein overcome the disadvantages described above. These, and other advantages, are provided by, for example, a method performed by one or more processors associated with one or more computing devices. The method accesses, by one or more of the processors, one or more data streams from a plurality of sensors, the sensors comprising an accelerometer and heart-rate monitor worn by an individual, in which the data streams include accelerometer data of the individual from the accelerometer and heart-rate data of the individual from the heart rate monitor, and self-reported demographic data of the individual collected by manual input by the individual, a first data set from the sensor data streams collected upon initial usage by the individual, the person being engaged in activity monitoring for the first time, and data received from continuous monitoring of the sensor data streams collected subsequent to baseline. The method detects if certain thresholds are met indicating the person is likely to have suffered a traumatic event where they need assistance, detects and monitors, by one or more of the processors, a set of trigger conditions comprising baseline accelerometer data and baseline heart-rate data, compared with current real time accelerometer data and heart-rate data, in which the baseline data measures normal activity, and in which the real time data is monitored for thresholds noting traumatic event likelihood. The method determines, by one or more of the processors, a current “Event Detection” profile of the person based on the analysis of the first data set (initial baseline data) and second data set (indicating a traumatic event) with respect to each other.

These and other advantages are also provided by, for example, a passive event detection system for a traumatic event. The system includes an input configured to receive data collected by a plurality of sensors, a computer-readable storage medium configured to store computer-executable instructions, and a computer processor configured to execute the computer-executable instructions. The computer-executable instructions may include instructions for accessing, by one or more of the processors, one or more data streams from a plurality of sensors, the sensors comprising an accelerometer and heart-rate monitor worn by an individual, in which the data streams comprise accelerometer data of the individual from the accelerometer and heart-rate data of the individual from the heart rate monitor, and self-reported demographic data of the individual collected by manual input by the individual a first data set from the sensor data streams collected upon initial usage by the individual, the person being engaged in activity monitoring for the first time, and data received from continuous monitoring of the sensor data streams collected subsequent to baseline, detecting if certain thresholds are met indicating the person is likely to have suffered a traumatic event where they need assistance, detecting and monitoring, by one or more of the processors, a set of trigger conditions comprising baseline accelerometer data and baseline heart-rate data, compared with current real time accelerometer data and heart-rate data, wherein the baseline data measures normal activity, and in which the real time data is monitored for thresholds noting traumatic event likelihood, and determining, by one or more of the processors, a current “Event Detection” profile of the person based on the analysis of the first data set (initial baseline data) and second data set (indicating a traumatic event) with respect to each other.

### BRIEF DESCRIPTION OF THE FIGURES

FIG. 1 shows a block diagram of the passive event detection system for individuals to detect traumatic events using physiological and environmental factors, according to one aspect of this disclosure.

FIG. 2 shows a plurality of sensors that may provide data to the system, according to one aspect of this disclosure.

FIG. 3 is a flowchart showing a method for detecting a potentially incapacitating traumatic event, according to one aspect of this disclosure.

### DETAILED DESCRIPTION

The terms and descriptions used herein are set forth by way of illustration only and are not meant as limitations. Those skilled in the art will recognize that many variations are possible within the spirit and scope of the invention as defined in the following claims, and their equivalents, in which all terms are to be understood in their broadest possible sense unless otherwise indicated.

Described herein are embodiments of a system and method of passive and active alert system for individuals to detect traumatic events using physiological and environmental factors. Embodiments overcome the problems described above, namely that the system can function passively and create an event notification in the event that the individual is incapacitated and cannot independently alert necessary personnel. For example, embodiments monitor an individual's heart rate (through heart rate monitor data) and movement (through accelerometer data) to estimate when an individual may have suffered a traumatic event and is in need of assistance.

Embodiments described herein use physiologic and environmental sensors to detect and passively (without the input of the injured individual) create an event notification. Embodiments may utilize various processes and systems to act upon this event notification, including then notifying Emergency Medical Services (“EMS”) and/or a set of contacts defined by the user. While there are numerous applications for embodiments described herein, the following provides three illustrative examples: elderly individuals, physically and/or mentally impaired individuals, and individuals engaging in adventurous or “risky” activities. In each example, prompt medical care often makes the difference in avoiding a more serious injury, or even death.

Embodiments provide a new way to identify the occurrence of an event that may have caused traumatic injury to the user. A heart rate monitor and accelerometer worn by the user (and potentially other sensors) provide information which is processed by embodiments described herein to determine when a potentially traumatic event has occurred.

A purpose of embodiments of a passive and active alert system for individuals to detect traumatic events using physiological and environmental factors is to foster peace of mind among at-risk individuals and their loved ones. Embodiments passively monitor certain sensors, e.g., heart rate and accelerometer, worn by the user; based on signals from these sensors, embodiments can make educated assumptions about when a traumatic event occurs (“event”).

Upon identification of an event, embodiments of a passive event detection system for individuals to detect traumatic events using physiological and environmental factors would output an event notification. This event notification could then be acted upon, including then notifying EMS and/or a set of contacts defined by the user of the embodiments. The event notification could include specific information that would facilitate a swift and proper response, thereby reducing the chance that an event will go undetected. The user can also actively identify an event, but the passive feature of embodiments described herein sets the passive and active alert system for individuals to detect traumatic events using physiological and environmental factors apart from product offerings currently available. Further, the use of the heart rate sensor data allows the system to minimize false positive alerts (alerts in which an event is detected but an event did not actually occur). This false positive might occur if the system only relied on accelerometer data. For example, if the system was dropped but not worn by the individual, the accelerometer would register an event. However, the passive event detection system would examine the heart rate data to determine if the system was actually worn by the individual. For example, if no heart rate was detected, then the system may determine that the individual was not wearing the system. Therefore, the system may not output an event notification.

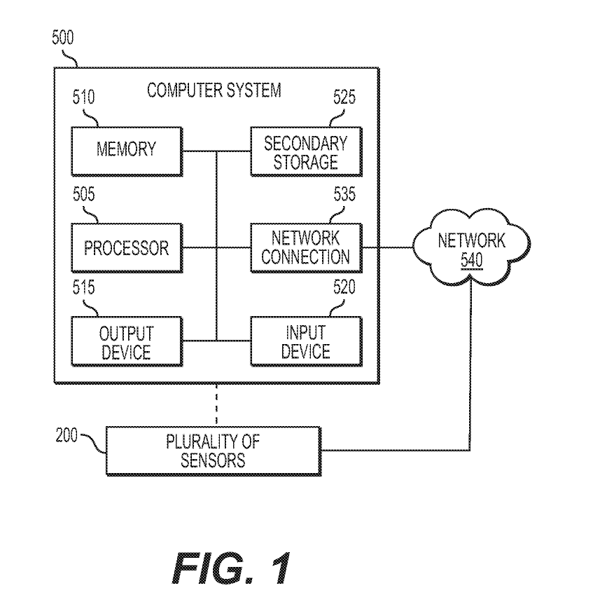

With reference now to FIG. 1, shown is a block diagram of a system illustrating exemplary hardware components for implementing embodiments of system and method for system and method of passive event detection for individuals to detect traumatic events using physiological and environmental factors. Computer system 500, or other computer system similarly configured may run method 100 (as described below) directly or indirectly through a subsystem component.

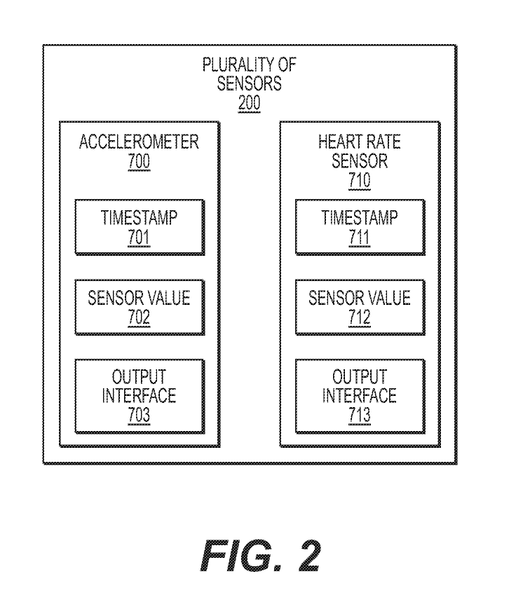

Computer system 500 typically includes a processor or processors 505 and memory 510, and may include an output device 515. Computer system 500 may contain an input device 520. Input device 520 would be used to receive sensor data, including but not limited to accelerometer sensor data 701 and 702 and heart rate sensor data 711 and 712, as described below. Computer system 500 may store input data on one or more database structures in secondary storage 525. Computer system 500 may also include a network connection 535, which may be connected to network 540. The network connection 535 which may be used, in addition to or in lieu of the input device 520, to receive sensor data such as accelerometer sensor data 701 and 702 and heart rate sensor data 711 and 712. The network connection 535 may also be used to transmit an event notification. One of ordinary skill in the art would readily recognize that any network 540, such as the Internet or Local Area Network (LAN), may be used. Computer system 500 may also be connected to a plurality of sensors 200 (shown in FIG. 2). The computer system 500 may be connected to the plurality of sensors 200 through a wired or a wireless connection. For example, if a wired connection is used, data generated by the plurality of sensors 200 may be received by the input device 520. If a wireless connection is used, data generated by the plurality of sensors 200 may be received by the network connection 535 via the network 540. In other aspects of this disclosure, at least one sensor of the plurality of sensors 200 may use a wired connection while at least another one of the sensors of the plurality of sensors 200 may use a wireless connection.

With reference now to FIG. 2, shown is exemplary embodiment of a plurality of sensors 200 that may provide data to computer system 500. Sensor 700 is an accelerometer and collects data including but not limited to a date and time stamp 701 and a sensor value 702. The sensor has an output device 703. An additional sensor 710 is a heart rate monitor and similarly collects data including but not limited to a date and time stamp 711 and a sensor value 712. The sensor has an output device 713. The sensor output devices 703, 713 may be used to output collected data to the computer system 500. For example, the output devices 703, 713 may transmit, using wired or wireless means, data collected to the input device 520 or to the network connection 535 via network 540. One of ordinary skill in the art would readily recognize that any number and any type of sensor may be utilized.

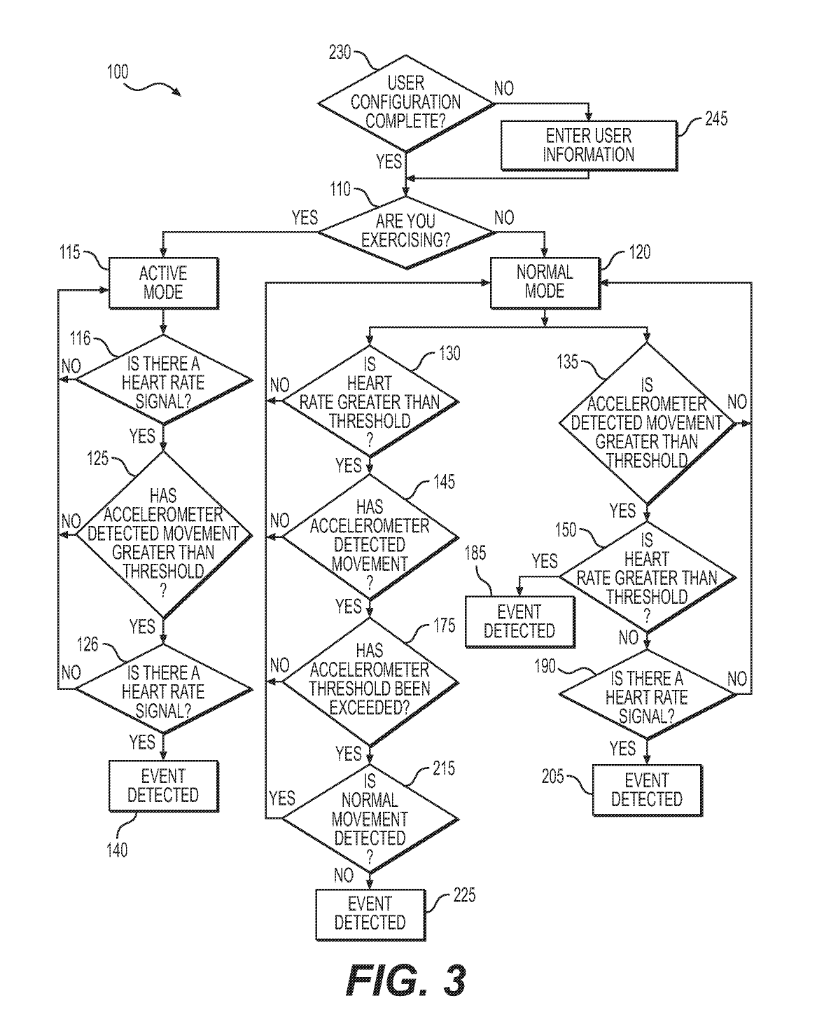

With reference now to FIG. 3, shown is exemplary embodiment of a method 100 for detecting a potentially incapacitating traumatic event. An embodiment uses a wearable accelerometer sensor 700 and heart rate sensor 710 (for example, a wrist-based smart watch or health-band). Embodiments include an algorithm, which based on certain data patterns observed in heart rate data and based on certain data patterns observed in accelerometer data, determines that an “event” has occurred. Examples of “events” are: falls, activity related incidents (e.g., bike crash, treadmill fall) or other traumatic events (e.g., car crash).

Upon starting method 100, an evaluation is made as to whether or not user configuration information 230 is complete. If it is not complete, block 245 is reached and the user is prompted with a series of questions, the answers to which comprise user information. Questions may include age, height, weight, and any known health conditions. When user information is complete, decision block 110 is reached, at which point the user selects normal mode 120 or active mode 115. Active mode 115 would be selected during exercise or otherwise strenuous periods of activity by the user, and normal mode 120 would be used at all other times.

Normal mode 120 is a primary mode for method 100. A primary mode selection would notify the algorithm that both the accelerometer sensor 700 data and the heart rate sensor 710 data should be evaluated by method 100, periodically or continuously, to determine if consecutive sensor data 701 and 702 reveal a change in acceleration that exceeds a threshold value. The threshold value may be set to any deviation from the baseline value. For example, the threshold may be a 50% change from the baseline accelerometer data or heart rate data. However, one of ordinary skill in the art would readily recognize that any deviation, such as 10% or 25%, from the baseline may be set as the threshold.

Upon changes in heart rate sensor data 712 that exceed the threshold shown in block 130, accelerometer sensor data 702 is evaluated for changes after threshold 130 was exceeded in block 145. If there are continued changes in sensor data 702 then these changes are compared to the threshold in block 175; changes in sensor data 702 which do not exceed the threshold in block 175 return method 100 back to normal mode 120. Changes in sensor data 702 which do exceed the threshold in block 175 are then subsequently analyzed in block 215 for normal motion. If normal motion is detected in block 215, then the method 100 may return to normal mode 120. If there are no continued changes in sensor data 702 then an event is detected in block 225.

Upon changes in accelerometer sensor data 702 that exceed the threshold in block 135, heart rate sensor 710 data is evaluated for consecutive sensor data 711 and 712 over a time period ranging from prior to exceeding the threshold in block 135 and until an event is either detected in decision block 185 or decision block 205, or the user returns to normal mode in decision block 120. Heart rate sensor 710 data that exceed the threshold in block 150 triggers event 185. Heart rate sensor 710 data that do not exceed the threshold in block 150 then evaluates for a heart rate signal block 190. Detection of a signal in block 190 triggers event detection in block 205. If heart rate signal in block 190 is not detected, then the user is returned to normal mode in block 120.

The method 100 may provide for an alternate mode if the wearer selects an active mode in block 115. A selection of the active mode 115 would notify the method 100 that heart rate sensor 710 data is expected to be higher than when in normal mode 120. Method 100 may, when in active mode 115, rely primarily on data from accelerometer sensor 700. A higher than normal heart rate is expected, and therefore the triggers for an event may be based on altered heart rate thresholds and/or may be based primarily on data from the accelerometer. In active mode 115 at decision blocks 116 and 126, the method 100 may evaluate heart rate sensor 710 data to ensure that a heart rate signal is detected. If no heart rate sensor 710 data is detected, the method 100 may return to active mode 115.

### Alternatives

Embodiments of the passive and active alert system for individuals to detect traumatic events using physiological and environmental factors may include a number of different sensors, operational modes, options and other features that may affect how the passive and active alert system operates. For example:
Sensors—utilize different, and/or additional sensors, including wearable, non-wearable, data-based, etc. A sensor typically measures a physical quantity and converts it into a signal that an observer or an instrument can read. For example, a mercury-in-glass thermometer converts a measured temperature into expansion and contraction of a liquid that can be read on a calibrated glass tube. A thermocouple converts temperature to an output voltage that a voltmeter can read. For accuracy, sensors are generally calibrated against known standards.
Algorithms included in the passive and active alert system may utilize additional sensor data points and logic to identify an event or events.
Embodiments may require the storage of sensor data before and after the event for later analysis.
Embodiments may require accessing our outputting sensor data for additional analysis or evaluation.
Algorithms included in the passive and active alert system may require monitoring longer periods of data.
The exact flow of decisions may be updated to improve accuracy and/or speed of event detection.
Embodiments may allow for variable and/or configurable settings, relating to threshold levels or other items
Processor and accompanying software may be external to, or bundled with, the sensors.

The terms and descriptions used herein are set forth by way of illustration only and are not meant as limitations. Those skilled in the art will recognize that many variations are possible within the spirit and scope of the invention as defined in the following claims, and their equivalents, in which all terms are to be understood in their broadest possible sense unless otherwise indicated.

1. A method comprising, by one or more processors associated with one or more computing devices:
accessing, by one or more of the processors, one or more data streams from a plurality of sensors, the sensors comprising an accelerometer and heart-rate monitor worn by an individual, wherein the data streams comprise:
accelerometer data of the individual from the accelerometer and heart-rate data of the individual from the heart rate monitor, and self-reported demographic data of the individual collected by manual input by the individual;
a first data set from the sensor data streams collected upon initial usage by the individual, the person being engaged in activity monitoring for the first time; and
data received from continuous monitoring of the sensor data streams collected subsequent to baseline;
detecting if certain thresholds are met indicating the person is likely to have suffered a traumatic event where they need assistance;
detecting and monitoring, by one or more of the processors, a set of trigger conditions comprising baseline accelerometer data and baseline heart-rate data, compared with current real time accelerometer data and heart-rate data, wherein the baseline data measures normal activity, and wherein the real time data is monitored for thresholds noting traumatic event likelihood;
determining, by one or more of the processors, a set of conditions wherein false positive event detection is avoided by using heart-rate data, or additional data, to determine when the system is in contact or worn by the individual; and
determining, by one or more of the processors, a current “Event Detection” profile of the person based on the analysis of the first data set (initial baseline data) and second data set (indicating a traumatic event) with respect to each other.

2. The method of claim 1, wherein the self-reported demographic data comprises one of age, height, weight, and any known health conditions.

3. The method of claim 1, further comprising selecting an active mode or a normal mode.

4. The method of claim 3, wherein a first threshold is associated with an active mode and a second threshold is associated with the normal mode.

5. The method of claim 4, wherein the first threshold is higher than the second threshold.

6. The method of claim 3, wherein when the current real time heart-rate data and the baseline heart-rate data are above the threshold values, determining movement based on the current real time accelerometer data.

7. The method of claim 6, wherein when no movement is determined, generating an alert noting a traumatic event likelihood.

8. The method of claim 6, wherein when movement is determined to have occurred, determining if the movement is normal, and wherein when the movement is determined not to be normal, generating an alert noting a traumatic event likelihood.

9. The method of claim 1, further comprising generating an event notification when a traumatic event is detected; and outputting the event notification to a third party.

10. A passive event detection system for a traumatic event, the system comprising:
an input configured to receive data collected by a plurality of sensors;
a computer-readable storage medium configured to store computer-executable instructions; and
a computer processor configured to execute the computer-executable instructions, the computer-executable instructions comprising instructions for:
accessing, by one or more of the processors, one or more data streams from a plurality of sensors, the sensors comprising an accelerometer and heart-rate monitor worn by an individual, wherein the data streams comprise:
accelerometer data of the individual from the accelerometer and heart-rate data of the individual from the heart rate monitor, and self-reported demographic data of the individual collected by manual input by the individual;
a first data set from the sensor data streams collected upon initial usage by the individual, the person being engaged in activity monitoring for the first time; and
data received from continuous monitoring of the sensor data streams collected subsequent to baseline;
detecting if certain thresholds are met indicating the person is likely to have suffered a traumatic event where they need assistance;
detecting and monitoring, by one or more of the processors, a set of trigger conditions comprising baseline accelerometer data and baseline heart-rate data, compared with current real time accelerometer data and heart-rate data, wherein the baseline data measures normal activity, and wherein the real time data is monitored for thresholds noting traumatic event likelihood;
determining, by one or more of the processors, a set of conditions wherein false positive event detection is avoided by using heart-rate data, or additional data, to determine when the system is in contact or worn by the individual; and
determining, by one or more of the processors, a current “Event Detection” profile of the person based on the analysis of the first data set (initial baseline data) and second data set (indicating a traumatic event) with respect to each other.

11. The system of claim 10, wherein the self-reported demographic data comprises one of age, height, weight, and any known health conditions.

12. The system of claim 10, wherein the computer-executable instructions further comprises:
selecting an active mode or a normal mode.

13. The system of claim 12, wherein a first threshold is associated with an active mode and a second threshold is associated with the normal mode.

14. The system of claim 13, wherein the first threshold is higher than the second threshold.

15. The system of claim 12, wherein when the current real time heart-rate data and the baseline heart-rate data are above the threshold values, the computer-executable instructions further comprise:
determining movement based on the current real time accelerometer data.

16. The system of claim 15, wherein when no movement is determined, the computer-executable instructions further comprise:
generating an alert noting a traumatic event likelihood.

17. The system of claim 15, wherein when movement is determined to have occurred, determining if the movement is normal, and wherein when the movement is determined not to be normal, the computer-executable instructions further comprise:
generating an alert noting a traumatic event likelihood.

18. The system of claim 10, the computer executable-instructions further comprise:
generating an event notification when a traumatic event is detected; and outputting the event notification to a third party.
